(19) United States
(12) Patent Application Publication
(10) Pub. No.: US 2019/0328315 A1
(43) Pub. Date: Oct. 31, 2019

(54) INTERNAL ANATOMICAL FORCE MEASUREMENT

(71) Applicants: Megan Wieser, Scottsdale, AZ (US); Jinglin Liu, Scottsdale, AZ (US); Amanda Smith, Scottsdale, AZ (US); Priscilla Hernandez, Scottsdale, AZ (US); Arizona Board of Regents on Behalf of Arizona State University, Scottsdale, AZ (US)

(72) Inventors: Jeffrey LaBelle, Tempe, AZ (US); Megan Wieser, Glendale, AZ (US); Jinglin Liu, Tempe, AZ (US); Amanda Smith, Tempe, AZ (US); Priscilla Hernandez, Providence, RI (US)

(21) Appl. No.: 16/396,258

(22) Filed: Apr. 26, 2019

Related U.S. Application Data
(60) Provisional application No. 62/672,348, filed on May 16, 2018.
(60) Provisional application No. 62/663,234, filed on Apr. 26, 2018.

(51) Int. Cl. A61B 5/00; G01L 1/14

(52) U.S. Cl. CPC A61B 5/4566; G01L 1/14; A61B 2562/0252; A61B 2562/0261; A61B 5/6885

(57) ABSTRACT

Systems, devices and methods involving static or active measurement of pressure, load, or other forces exerted on or between internal anatomical structures are described. A load and/or strain sensor, which may be implantable or otherwise deployed during a medical procedure, along with systems and methods thereof, are also described. These measurements techniques and sensors can provide real-time as well as accumulated data indicative of stresses or other forces being exerted on or between internal anatomical structures of a target patient, e.g., portions or interfaces of the skeletal system.

### CROSS-REFERENCE TO RELATED APPLICATIONS

This application claims priority to U.S. provisional application 62/663,234, which was filed on Apr. 26, 2018 and is entitled Intra-Operative Device to Measure Load on Spine Segments in the Lumbar Region and to U.S. provisional application 62/672,348, which was filed on May 16, 2018 and is entitled Dynamic Vertebrae Force Measurement. Each of the '234 application and the '348 application is incorporated in its entirety by reference into this application.

### TECHNICAL FIELD

This application relates generally to systems, devices and methods involving static or active measurement of pressure, load, or other forces in spinal vertebrae or other internal anatomically related force transfer and/or support interfaces. In particular, this application relates to systems and devices comprising a load and/or strain sensor, which may be implantable within the body of a patient, and related systems and methods thereof.

### TECHNICAL BACKGROUND

Scoliosis, which can be defined as a curvature of the back that is 10 degrees or greater as measured by the Cobb angle, affects 2-3% of the population, or approximately 6-9 million patients in the U.S. Treatments include: observation, brace, and surgery, including fusion surgery. The severity of the curvature determines the type of treatment used, with the most severe curvatures treated through surgery. Approximately 38,000 patients receive surgery per year.

There are about 29,000 Adolescent Idiopathic Scoliosis (AIS) surgeries performed per year, each costing about $92,000 at the time of writing. In addition to scoliosis applications, spinal fusion surgery may be an alternative for spine stabilization, deformation correction, and back pain relief.

Current spinal surgeries depend solely on the technique and experience of the surgeons, who rely on visual inspection and intuition to measure the load and/or pressure attributable to misaligned, fused or otherwise surgically modified vertebrae. Even more advanced visual inspection technologies, such as X-ray radiography, computed tomography (CT), magnetic resonance imaging (MRI), and Intraoperative neurophysiological monitoring (TOM), also involve a significant amount of inaccuracy and uncertainty.

### BRIEF SUMMARY

Embodiments may include an implantable load and/or pressure sensor and related methods and processes. The sensor may be implanted and used during surgical procedures or afterwards in order to determine forces being exerted on the vertebrae or other structural anatomical systems. Through real-time or stored data acquired by a deployed sensor of embodiments, or a sensor disposed on or in a surgical device of embodiments, a medical provider can receive information indicative of forces being experienced at a specific anatomical interface. Through this additional information a medical provider may make decisions during a medical procedure or afterwards. These decisions may include positioning decisions for a support being implanted into the body of a patient during a procedure, material selections during a procedure, and device selections during a procedure. The decisions may also be made after the sensor is implanted or otherwise deployed and the procedure is completed. These monitoring type decisions can include when a degradation of an anatomical interface is needing repair and for prophylactic reasons as well.

Sensors of embodiments may comprise compressible materials positioned between conductive plates where the capacitance of the materials changes under known parameters such that compression of the sensor can be determined by changes in an observed capacitance of the sensor. The compressible materials may be chemically treated to promote a determinable relationship between capacitance change and compression of the materials. In some embodiments, the compressible materials may be surrounded in whole or in part by electrically insulative materials. These insulative materials may be provided to reduce harm to any anatomy near the implanted sensor, to better contain the accumulated electrons between the conductive plates, for each reason, and for other reasons as well. Forces acting on various portions of the body may be sensed with embodiments and this sensing may be conducted in real-time as well as be stored and used at a later time, after initial sensing.

A sensor, or multiple sensors, may be used to measure forces on the spine or other anatomical systems where mechanical forces are transferred or develop. Forces may be measured across the whole spine, across a region of the spine, or between individual pairs of vertebral discs of the spine. Likewise, forces may be measured across the whole joint, across a region of a joint, or between individual bones or other interfaces of the anatomy as well as combinations thereof.

In use, an exemplary sensor, or multiple sensors, may be used before alignment surgery, such as when evaluating a patient's condition; may be used during alignment surgery, such as when adjusting a patient's spine or other targeted disfunction, and/or when placing implantable devices; and may be used after alignment surgery, such as during rehabilitation, as well as after rehabilitation, as well as combinations thereof. As noted, an exemplary sensor, or multiple sensors, may be used outside of surgery as well, for example, when evaluating a patient's spine or other malfunctioning anatomical support and monitoring any changes thereof.

A sensor or multiple sensors may be integrated into surgical tools, such as gloves, spreaders, screws, couplers, drills, saws, forceps, etc. In some embodiments, during or after a procedure, a capacitive load and/or strain sensor may be employed to measure developed forces at a specific anatomical interface. For example, sensors may employ a deformable foam that may be calibrated in order to measure a passive or active load on spine segments in and/or between the thoracolumbar and/or lumbar region. Other regions of the anatomy may be measured using embodiments as well. These regions can include the thoracolumbar region, the various joints of the body, and the thoracic cavity. Embodiments may provide measurement on a real-time basis as well as store measured results for subsequent use and analysis. These measurements and ongoing monitoring may be provided in quantitative form.

As noted above, embodiments may include a load and/or strain sensor with electrochemical properties that may measure load and/or strain at various regions of the body. In some embodiments, a redox reaction within a sensor may be able to power the sensor, provide a voltage potential, provide other electrical purposes while the foam may be calibrated and measurable in order to quantitatively measure a change of load and/or strain experienced by the sensor. Thus, in embodiments, the sensor system may provide providers with a quantitative way to measure the load and/or strain of targeted regions of the body, instead of having a surgeon or other provider rely entirely on visual inspection and intuition. Thus, sensor systems of embodiments may be used preoperatively, intraoperatively, and/or post-operatively to measure the load and/or strain distribution in the thoracolumbar region, in the lumbar region, between these regions, and in other regions of the body as well.

Embodiments may comprise a micro-electromechanical load/strain sensing system, an environmental factor controlling algorithm system, or both. Firmware of embodiments may take into considerations factors such as hysteresis, non-repeatability, temperature, creep, force feedback due to various bone density, or combinations thereof when providing results or making other determinations. In some embodiments, the sensor system may numerically measure load and/or strain with the percentage accuracy increasing with signaling calibration, in comparison with non-calibrated signal. Calibration can provide an accuracy increase of 7% as well as a larger or smaller percentage in embodiments.

In embodiments, a sensor system may comprise a sensor connected in a wire or wireless manner to a patch. The sensor may be implantable, while the patch may be extra-corporal. A redox reaction within a sensor may power the sensor, a battery connected in a wired or wireless manner to the sensor may power the sensor, or the sensor may be powered by other means as well. In embodiments, a sensor may comprise an electrode coated entirely, partially, or in segments, with a foam where the foam may comprise or be impregnated with electroactive moieties. In embodiments, a sensor may comprise electrically conductive plates having foam therebetween, where the foam may comprise or be impregnated with electroactive moieties. The sensor may distinguish change in load and/or strain using a voltage provided by a chemical reaction contained in the sensor. The sensor may distinguish change in load and/or strain using change in resistance, a change in capacitance, a movement of electroactive moieties within the foam, or a combination thereof. The sensor, and/or a patch coupled to a sensor, may comprise a wireless communication device, which may be used to communicate in one or both directions with a device, such as a cell phone, a computer, or a dedicated device to report changes in loads and/or strains experienced by an implanted sensor or grouping of sensors. The device may comprise firmware and/or software capable of converting the raw data from the sensor into Newtons or other measure of force. The device may have a screen for displaying raw and/or converted data.

In embodiments, the load sensor may comprise conductive plates disposed approximately parallel to one another around opposite sides of a compressible foam sheet, the compressible foam sometimes comprising electroactive material and/or sometimes being impregnated with electroactive moieties. The plates may comprise metal, such as copper. The sensor may comprise biocompatible material, may be encased in biocompatible material, or both. The sensor may be connected by a wire to a controller patch. The controller patch may be connected via wired or wireless connection to a device. The device, the patch, the sensor, or each may comprise at least one of (i) one or more potentiostat, (ii) one or more Analog-to-Digital converter (ADC), (iii) one or more microprocessor, (iv) one or more microcontroller, and (v) one or more operational amplifiers. The sensor, device, and/or patch of embodiments may also each comprise a power source. The sensor, device, and/or patch may comprise a wireless communication device and may communicate via wired and/or wireless techniques. The device, and the patch, may each comprise a display and/or an indicator.

Embodiments may include methods for measuring a force exerted on a spine. Methods may comprise processes of: (a) implanting one or more electrochemical load sensor in one or more vertebral discs, the sensor comprising: (i) an electrode; (ii) the electrode at least partially encased in a compressible foam; and (iii) the compressible foam comprising or being impregnated with electroactive moieties, the electroactive moieties having a first distribution in an uncompressed state; (b) supplying a current to the electrode; (c) exerting a load on the spine, thereby compressing the foam, thereby changing the distribution of electroactive moieties of the foam to a second distribution; and (d) measuring the load by measuring the change in current in the electrode caused by the change in distribution of electroactive materials of the foam.

Methods may similarly comprise processes of: (a) providing an electrochemical load sensor, the sensor comprising: a capacitive load cell comprising two conductive plates comprising a first compressible foam therebetween, the compressible foam comprising or being impregnated with electroactive moieties, the capacitive load cell having a first capacitance in an uncompressed state; (b) applying a voltage to at least one plate; (c) exerting a mechanical load on the capacitive load cell, thereby compressing the foam, thereby changing the capacitance of the load cell to a second capacitance; and (d) measuring the mechanical load by measuring the change in capacitance caused by the change in thickness of the foam caused by an applied compressive force, wherein the change in capacitance is proportional or otherwise related to the change in thickness of the foam.

Embodiments may include an anatomical force sensor system that comprises a first electrically conductive plate and a second electrically conductive plate, the first conductive plate and the second conductive plate may be spaced a distance apart from each other and not touching each other. A first compressible foam disposed between the first electrically conductive plate and the second electrically conductive plate may also be provided. The first compressible foam may have a first thickness in a first compression state and a second thickness in a second compression state. In some embodiments, a second compressive foam may be disposed about the first compressive foam, the second compressive foam may provide electrical insulation to the first compressive foam and a voltage source may be electrically coupled to the first electrically conductive plate and the second electrically conductive plate. In some embodiments the voltage source may be configured to provide a voltage potential between the first conductive plate and the second conductive plate. A controller may also be provided. The controller may be configured to sense changes in capacitance between the first electrically conductive plate and the second electrically conductive plate when the conductive plates are placed within a body of a patient. In embodiments, these sensed changes in capacitance may be used to determine a change in distance between the first conductive plate and the second conductive plate.

Other embodiments may also be provided. These may include the first compressible foam and the second compressible foam may each having a top and a bottom and at least one side, the at least one side of the first compressible foam may be surrounded by the second compressible foam. Still further, the top of the first compressible foam and the top of the second compressible foam may be adjacent to the first electrically conductive plate and the bottom of the first compressible foam and the bottom of the second compressible foam may be adjacent to the second electrically conductive plate. Also, the top of the first compressible foam and the top of the second compressible foam may touch the first electrically conductive plate and the bottom of the first compressible foam and the bottom of the second compressible foam may touch the second electrically conductive plate. In certain embodiments, the first compressible foam may be cylindrical and the second compressible foam may be polygonal. In certain embodiments, the voltage source may be electrically coupled to the first electrically conductive plate with a wire and electrically coupled to the second conductive plate with a wire. Still further, in certain embodiments, the first compressible foam may comprise electroactive moieties, wherein the first electrically conductive plate and the second electrically conductive plate may be parallel, and wherein the voltage source may be sized to limit a capacitive charge of no greater than 100 millivolts from being developed between the first electrically conductive plate and the second electrically conductive plate. In certain embodiments, the controller may be further configured to determine, using the change in distance between the first electrically conductive plate and the second electrically conductive plate, real-time compressive forces experienced by the first electrically conductive plate or the second electrically conductive plate or both. In certain embodiments, the first electrically conductive plate, the second electrically conductive plate, the first compressible foam, and the second compressive foam may be resident within an implantable biocompatible sensor. In certain embodiments, the first electrically conductive plate, the second electrically conductive plate, the first compressible foam, the second compressive foam, and the voltage source, may be resident within an implantable biocompatible sensor.

Certain embodiments may comprise an implantable sensor and a controller where the implantable sensor may comprise a first electrically conductive plate, and a second electrically conductive plate. The first electrically conductive plate and the second electrically conductive plate may be spaced a distance apart from each other and may not touch each other. In certain embodiments, a first compressible foam may be disposed between the first electrically conductive plate and the second electrically conductive plate, the first compressible foam comprising electroactive moieties, the first compressible foam having a first thickness in a first compression state and a second thickness in a second compression state; and a second compressive foam may be disposed about the first compressive foam, the second compressive foam providing electrical insulation to the first compressive foam. In certain embodiments, the controller may be configured to sense changes in capacitance between the first electrically conductive plate and the second electrically conductive plate and use these sensed changes in capacitance to determine a real-time change in distance between the first electrically conductive plate and the second electrically conductive plate when the conductive plates are resident within a body of a patient.

Certain embodiments may comprise a voltage source electrically coupled to the first electrically conductive plate and the second electrically conductive plate, the voltage source may be configured to provide a voltage potential between the first electrically conductive plate and the second electrically conductive plate. In certain embodiments, the first compressible foam and the second compressible foam may each have a top and a bottom and at least one side, the at least one side of the first compressible foam may be surrounded by the second compressible foam. In certain embodiments, the top of the first compressible foam and the top of the second compressible foam may be adjacent to the first electrically conductive plate and the bottom of the first compressible foam and the bottom of the second compressible foam may be adjacent to the second electrically conductive plate. In certain embodiments, the first compressible foam may comprise electroactive moieties, wherein the first electrically conductive plate and the second electrically conductive plate may be parallel, and wherein the voltage source may be sized to limit a capacitive charge of no greater than 1,000 millivolts from being developed between the first electrically conductive plate and the second electrically conductive plate. In certain embodiments, the controller may be further configured to determine, using the change in distance, real-time compressive forces experienced by the first electrically conductive plate or the second electrically conductive plate or both.

Embodiments may also comprise a method for measuring forces exerted on one or more vertebrae. These embodiments may include receiving a data signal at a controller and providing a determination of real-time force exerted on an implanted anatomical sensor. The data signal may be generated by a sensor positioned adjacent one or more vertebrae of a patient and the implanted anatomical sensor may comprise a first electrically conductive plate, a second electrically conductive plate. In certain embodiments, the first conductive plate and the second conductive plate may be spaced a distance apart from each other and not touching each other. Certain embodiments may also comprise a first compressible foam disposed between the first electrically conductive plate and the second electrically conductive plate, the first compressible foam comprising electroactive moieties, the first compressible foam having a first thickness in a first compression state and a second thickness in a second compression state; and a second compressive foam disposed about the first compressive foam, the second compressive foam providing electrical insulation to the first compressive foam. In certain embodiments, the controller may be configured to sense changes in capacitance between the first electrically conductive plate and the second electrically conductive plate and may use these sensed changes in capacitance to determine a change in distance between the first conductive plate and the second conductive plate.

Various features, steps, processes, components, and subcomponents, as may be employed in embodiments, are provided herein. These features, steps, processes, components, subcomponents, partial steps, systems, devices, etc. may be adjusted, combined and modified in various fashions and various ways among and between the teachings and figures provided herein, as well as in other ways not specifically described herein but consistent with the teachings and discussion of this disclosure.

### BRIEF DESCRIPTION OF THE DRAWINGS

The accompanying drawings illustrate embodiments.

FIG. 1A shows an exemplary electrochemical sensor; FIG. 1B shows the sensor without a covering plate to view foam; and FIG. 1C shows a schematic of applied axial compression forces that may act upon a sensor, each as may be employed in accordance with some embodiments.

FIG. 2 shows multiple sensors, a voltage source, and a controller, as may be employed in accordance with some embodiments.

FIG. 3 shows system-level schematics of communication flows as may be employed in accordance with some embodiments.

FIG. 4 shows an exemplary under-screw cap sensor and a rod and screw anchor, as may be employed in accordance with some embodiments.

FIG. 5 shows a breakdown of mechanical loading that may be measured, as may be employed in accordance with some embodiments.

FIG. 6 shows an exemplary schematic of operational processes and components of a sensor system, as may be employed in accordance with some embodiments.

FIG. 7 shows an exemplary schematic of operational processes and components, as may be employed in accordance with some embodiments.

### DETAILED DESCRIPTION

This application relates generally to systems, devices and methods involving active measurement of pressure, load, or other forces between spinal vertebrae or other internal anatomically related force transfer and/or support interfaces. In particular, this application relates to systems and devices comprising a load and/or strain sensor, which may be implantable or otherwise deployed during a medical procedure, and related systems and methods thereof. Embodiments may include an anatomical force sensor system that comprises a first electrically conductive plate and a second electrically conductive plate, the first conductive plate and the second conductive plate may be spaced a distance apart from each other and not touching each other. A first compressible foam disposed between the first electrically conductive plate and the second electrically conductive plate may also be provided. The first compressible foam may have a first thickness in a first compression state and a second thickness in a second compression state. In some embodiments, a second compressive foam may be disposed about the first compressive foam, the second compressive foam may provide electrical insulation to the first compressive foam and a voltage source may be electrically coupled to the first electrically conductive plate and the second electrically conductive plate. In some embodiments the voltage source may be configured to provide a voltage potential between the first conductive plate and the second conductive plate. A controller may also be provided. The controller may be configured to sense changes in capacitance between the first electrically conductive plate and the second electrically conductive plate when the conductive plates are placed within a body of a patient. In embodiments, these sensed changes in capacitance may be used to determine a change in distance between the first conductive plate and the second conductive plate.

Other embodiments may also be provided. These may include the first compressible foam and the second compressible foam may each having a top and a bottom and at least one side, the at least one side of the first compressible foam may be surrounded by the second compressible foam. Still further, the top of the first compressible foam and the top of the second compressible foam may be adjacent to the first electrically conductive plate and the bottom of the first compressible foam and the bottom of the second compressible foam may be adjacent to the second electrically conductive plate. Also, the top of the first compressible foam and the top of the second compressible foam may touch the first electrically conductive plate and the bottom of the first compressible foam and the bottom of the second compressible foam may touch the second electrically conductive plate. In certain embodiments, the first compressible foam may be cylindrical and the second compressible foam may be polygonal. In certain embodiments, the voltage source may be electrically coupled to the first electrically conductive plate with a wire and electrically coupled to the second conductive plate with a wire. Still further, in certain embodiments, the first compressible foam may comprise electroactive moieties, wherein the first electrically conductive plate and the second electrically conductive plate may be parallel, and wherein the voltage source may be sized to limit a capacitive charge of no greater than 100 millivolts from being developed between the first electrically conductive plate and the second electrically conductive plate. In certain embodiments, the controller may be further configured to determine, using the change in distance between the first electrically conductive plate and the second electrically conductive plate, real-time compressive forces experienced by the first electrically conductive plate or the second electrically conductive plate or both. In certain embodiments, the first electrically conductive plate, the second electrically conductive plate, the first compressible foam, and the second compressive foam may be resident within an implantable biocompatible sensor. In certain embodiments, the first electrically conductive plate, the second electrically conductive plate, the first compressible foam, the second compressive foam, and the voltage source, may be resident within an implantable biocompatible sensor.

Certain embodiments may comprise an implantable sensor and a controller where the implantable sensor may comprise a first electrically conductive plate, and a second electrically conductive plate. The first electrically conductive plate and the second electrically conductive plate may be spaced a distance apart from each other and may not touch each other. In certain embodiments, a first compressible foam may be disposed between the first electrically conductive plate and the second electrically conductive plate, the first compressible foam comprising electroactive moieties, the first compressible foam having a first thickness in a first compression state and a second thickness in a second compression state; and a second compressive foam may be disposed about the first compressive foam, the second compressive foam providing electrical insulation to the first compressive foam. In certain embodiments, the controller may be configured to sense changes in capacitance between the first electrically conductive plate and the second electrically conductive plate and use these sensed changes in capacitance to determine a real-time change in distance between the first electrically conductive plate and the second electrically conductive plate when the conductive plates are resident within a body of a patient.

Certain embodiments may comprise a voltage source electrically coupled to the first electrically conductive plate and the second electrically conductive plate, the voltage source may be configured to provide a voltage potential between the first electrically conductive plate and the second electrically conductive plate. In certain embodiments, the first compressible foam and the second compressible foam may each have a top and a bottom and at least one side, the at least one side of the first compressible foam may be surrounded by the second compressible foam. In certain embodiments, the top of the first compressible foam and the top of the second compressible foam may be adjacent to the first electrically conductive plate and the bottom of the first compressible foam and the bottom of the second compressible foam may be adjacent to the second electrically conductive plate. In certain embodiments, the first compressible foam may comprise electroactive moieties, wherein the first electrically conductive plate and the second electrically conductive plate may be parallel, and wherein the voltage source may be sized to limit a capacitive charge of no greater than 1,000 millivolts from being developed between the first electrically conductive plate and the second electrically conductive plate. In certain embodiments, the controller may be further configured to determine, using the change in distance, real-time compressive forces experienced by the first electrically conductive plate or the second electrically conductive plate or both.

Embodiments may also comprise a method for measuring forces exerted on one or more vertebrae. These embodiments may include receiving a data signal at a controller and providing a determination of real-time force exerted on an implanted anatomical sensor. The data signal may be generated by a sensor positioned adjacent one or more vertebrae of a patient and the implanted anatomical sensor may comprise a first electrically conductive plate, a second electrically conductive plate. In certain embodiments, the first conductive plate and the second conductive plate may be spaced a distance apart from each other and not touching each other. Certain embodiments may also comprise a first compressible foam disposed between the first electrically conductive plate and the second electrically conductive plate, the first compressible foam comprising electroactive moieties, the first compressible foam having a first thickness in a first compression state and a second thickness in a second compression state; and a second compressive foam disposed about the first compressive foam, the second compressive foam providing electrical insulation to the first compressive foam. In certain embodiments, the controller may be configured to sense changes in capacitance between the first electrically conductive plate and the second electrically conductive plate and may use these sensed changes in capacitance to determine a change in distance between the first conductive plate and the second conductive plate.

As noted above, in some embodiments, devices, systems and processes, may serve to minimize or avoid complications of the surgery by providing accurate and/or real-time measurement of applied forces and resultant pressures at various locations in the body. In some embodiments, devices, systems, and processes can translate contact forces into an applied load and/or strain display to a surgeon, technician, researcher, or other medical practitioner for a specific location of the body. This display of real-time loading can be provided during surgery as well as afterwards.

As noted above, embodiments may include a load and/or strain sensor and related methods and systems. The sensor may be implantable and/or may be integrated into surgical tools, such as gloves, spreaders, screws, couplers, drills, saws, forceps, etc. In some embodiments a capacitive load and/or strain sensor may be employed to measure developed forces at a specific anatomical interface. An exemplary sensor may employ a deformable foam that may be calibrated in order to measure a passive or active load on spine segments in and/or between the cervical, thoracic, thoracolumbar, and/or lumbar region. Other regions of the anatomy may be measured using embodiments as well. These regions can include the thoracolumbar region, the various joints of the body, and the thoracic cavity. Embodiments may provide measurement on a real-time basis as well as store measured results for subsequent use and analysis. These measurements and ongoing monitoring may be provided in quantitative form.

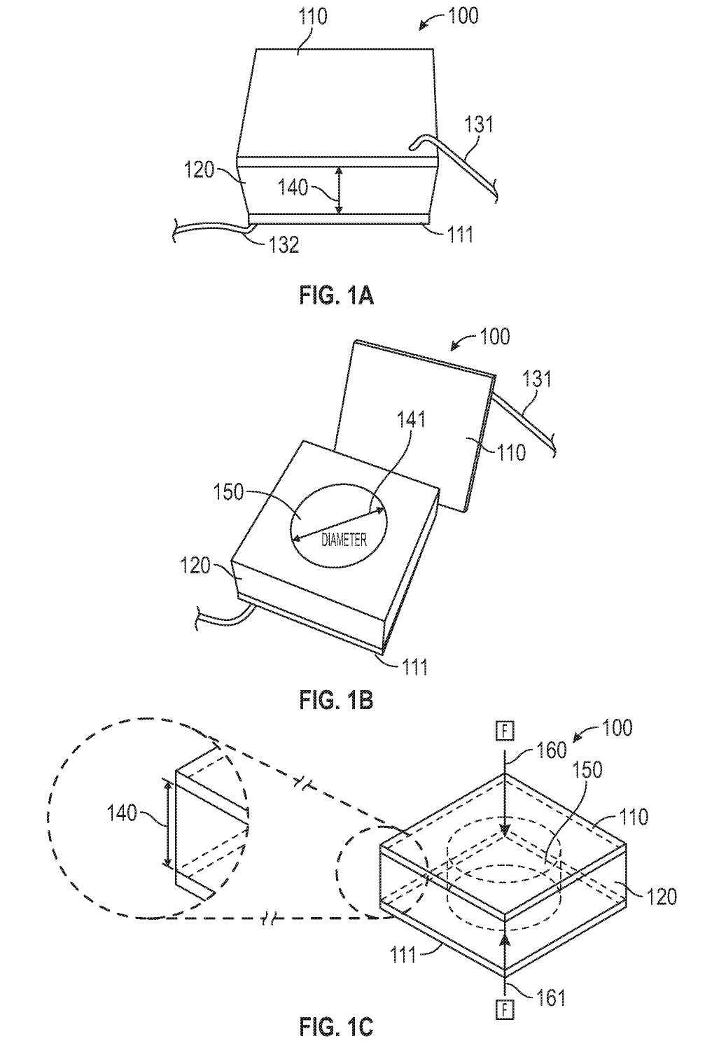

In embodiments, a sensor system may comprise a capacitive and/or electrochemical load sensor, an electrode and/or wire, a patch, an external device, or combinations thereof. In embodiments, an electrochemical load sensor system may comprise one or more of the following: an electrode or wire partially encased, fully encased, or encased in regions or sections, by a first “foam”, which may comprise electroactive material and/or or be impregnated with electroactive moieties. The first foam may comprise electroactive material and/or be impregnated with electroactive moieties. In embodiments, the outside (i.e., the side not contacting the electrode) of the first foam may be partially or entirely encased in or surrounded by a second foam, which may not comprise electroactive material and/or or be impregnated with electroactive moieties. The second foam may comprise material with electrical insulating properties. In embodiments, the capacitive and/or electrochemical load sensor may be planar in shape and may comprise conductive plates disposed approximately parallel to one another on two sides of a sheet comprising a first foam. The first foam may be partially or entirely surrounded by a second foam. The first foam may comprise electroactive material and/or or be impregnated with electroactive moieties while the second foam in this or other embodiments may not comprise electroactive material and/or or be impregnated with electroactive moieties and may have insulative properties. The conductive plates may comprise metal, such as copper, gold, and/or platinum. The plates and/or first and second foams may be shaped and/or arranged so that the first foam contacts part of all of each plate. See, e.g., FIGS. 1A-1C. Such embodiments may be referred to as “plate-foam-plate arrangements”.

The sensor may comprise an electrode partially or entirely coated in foam and/or may comprise a plate-foam-plate arrangement. The force range requirement of the sensor for may be approximately 240-500N. A first foam may comprise or be impregnated with electroactive moieties. A second foam may fully or partially surround or encapsulate the first foam. The second foam may have insulating properties. The sensor may be connected via a wired or wireless connection to an external device, such as a patch, a cell phone, computer, or dedicated device, which may provide power to the sensor, may collect current, signal, or other data from the sensor, may convert data from the sensor to Newtons other measure of force, and may display the raw or converted data. The external device may comprise a display, which may display raw or processed data from the sensor. The external device may comprise an alarm, light, or other indicator, which may be used to signal the practitioner when a certain force has been met or exceeded. The external device may receive power from a battery or from an A/C source. After use, any wire connection from the sensor may be cut or otherwise removed, allowing the sensor, which may comprise biocompatible materials, to remain in place.

The first and/or second foams may be compressible, preferably linearly compressible, i.e., the foams deform in a predictable and linear way when exposed to a linear load, and may be comprised of gels, hydrogel, compressible plastic, polymers, polyurethane, conductive thermoset, or thermoplastic elastomer, or combinations thereof. The first and/or second foam may be chosen or optimized to improve reliability and/or repeatability, such as by reducing material fatigue and increasing mechanical strength and these characteristics may be adjusted depending on the application in which they are being used.

The electroactive material may comprise a highly conductive material and can include highly conductive particles having a diameter of approximately 1-20 nm. Examples of electroactive materials may also include electrolytes, nanoparticles, multiwalled or single walled carbon nanotubes, graphite, ions, small molecules, conductive powders, nanomaterials, quantum dots, ferrocyanide, ferricyanide, Potassium Ferricyanide (III), electron mediators, redox probes, metal powders, carbon powders, conductive powders, and combinations thereof.

In some embodiments, the first foam may be provided with an electrical current via the wire, the electrode, and/or one or both of the plates. Compression of the first foam may change the resistance characteristics of the foam, may change the concentration of electroactive material in contact with the electrode and/or wire, or change both. In embodiments, such as plate-foam-plate arrangements, compression of the first foam may change the thickness of the foam and the distance between the plates, thereby changing the capacitance, where change in capacitance is proportional to change in thickness. Such changes may be measured as a change in current, which may be transmitted via the electrode and/or wire or which may be converted to voltage or another signal by a device, such as a potentiostat, which may be included in the sensor system, and transmitted as voltage or data.

Electrochemical load sensors of embodiments may comprise other data-processing hardware, firmware, or software, such as an Analog-to-Digital converter (ADC), a microprocessor, a microcontroller, an operational amplifier, or combinations thereof. In embodiments, where a patch is used, data may be sent from the sensor to the patch in an electrical form either in analog or digital form. Where a patch is not used, data may be sent from the sensor to a cell phone, computer, or dedicated device in the form of current, voltage, or other data, in any state of analog or digital processing.

In embodiments, the sensor, the electrode, the wire, the plates, the first foam, the second foam, and/or the electroactive moieties, and/or other components, may comprise or be coated with biocompatible material(s). “Biocompatible material(s)” as used herein may refer to materials that are safe to be implanted in a patient's body, including materials that are safe for temporary, but not permanent, implantation. “Biocompatible material(s)” as used herein may refer to materials that do not cause physical trauma or that cause minimal physical trauma; materials that are non-toxic or of low toxicity; materials that are not physiologically reactive or are minimally physiologically reactive; and/or materials that are not immunologically reactive or are minimally immunologically reactive. “Biocompatible material(s)” as used herein may refer to materials that have the ability to perform with an appropriate host response. “Biocompatible material(s)” as used herein may refer to materials that are or can be approved by the Food and Drug Administration (“FDA”).

In embodiments, sensors may comprise an outer coating surrounding or partially surrounding the sensor. The coating material may be biocompatible. The coating may be impervious to the foam and/or electroactive material, thereby preventing or reducing contact of the plates, foam, and/or electroactive material and/or other components with the surrounding tissue. The sensor may be designed to minimize output of heat.

In embodiments, sensors may be sized to be implanted via needle or cannula, although larger sizes requiring surgical implantation are also contemplated.

In embodiments, the foam of the sensor may be compressed by bodily movements when implanted. Sensors and combinations of sensors may detect various types of movement, such as back and forth movement, side-to side movement, rotational movement, and the like. Sensors and combinations of sensors may be used to measure various parameters, such as force, pressure, load distribution, and the like.

In embodiments, a sensor or combination of sensors may be attached by wire or wirelessly to an external monitoring patch, which may remain outside a patient's body. The wire may comprise a conductive or highly conductive material. The wire may comprise metal, such as copper, gold, and/or platinum. The wire may comprise carbon. The wire may be of an appropriate diameter to be inserted by needle or cannula, although larger diameters are also contemplated. The wire may be of a length suitable to attach a sensor implanted in a patient to a patch on the outside of the patient. Lengths in the range of inches, such as approximately 1-2-3 inches are contemplated. Where an external monitoring patch is not used, where sensors may be attached directly by wire to a cell phone, computer or dedicated device, the wire may be longer. The wire may be made of or coated with biocompatible material(s). The wire may provide current to the sensor, carry current from the sensor, carry a signal from the sensor, carry data from the sensor, provide an attachment or tether for removal of the sensor, or combinations thereof, or other functionalities.

The patch may be made of material which preferably will be minimally irritating to the patient, but suitable for containing or attaching necessary or desired components. The patch may be suitably sized to achieve a balance between comfort for the patient and inclusion of necessary or desired components. The patch may be attached to the patient's body by an adhesive, which may be biocompatible but not easily subject to unintentional removal. Examples of such adhesives include polyolefin tape, acrylic, and medical grade adhesive. The patch may be attached to the patient's body by mechanical means, such as a strap.

In some embodiments, the sensor may comprise or be electrically coupled to a power source. The power source may be designed to minimize output of heat. Also, the patch may comprise a power source. The patch may comprise a device for receiving power wirelessly from an external device, such as a cell phone or dedicated device. The patch may comprise a device for converting bodily motion to electrical power. The patch may comprise a device for converting light to electrical power. The patch may comprise a battery. The battery may be a small battery, such as a wafer battery, of which the following are examples: CR2025, CR2016, TL2450, TL5934, TL5134. The patch may comprise a device to receive power wirelessly. The patch may employ resonant inductive coupling, wherein a dual coil system may operate at resonance frequency and may create a magnetic field that transfers energy from a transmitting coil to a receiving coil. The patch may be designed for low-power dissipation. The patch may maintain enough power to allow the sensor(s) that it powers to work properly and with precision. The patch may be designed to minimize output of heat.

The sensor and/or patch may comprise a wired and/or wireless device for sending and/or receiving data to and/or from an external device, such as a cell phone, computer, or a dedicated device in a home or clinical setting. The sensor and/or patch may comprise a transceiver. The sensor and/or patch may comprise a Bluetooth device. The sensor, the patch, or the external device may comprise a potentiostat, which may convert a current signal to a voltage signal. The sensor, the patch, and/or the external device may comprise other data-processing hardware, firmware, or software, such as an Analog-to-Digital converter (ADC), a microprocessor, a microcontroller, an operational amplifier, or combinations thereof. The sensor, the patch, and/or the external device may comprise hardware, firmware, and/or software for converting the signal or raw data from the sensor or patch into a more useful format, such as Newtons or other measurement of force. Data may be sent from the sensor and/or patch to the external device in the form of current, voltage, or other data, in any state of processing. The sensor, patch, and/or external device may comprise a display for displaying the raw and/or converted data. The sensor, patch, and/or external device may comprise a computer or phone display, an LED display, and e-ink display, or other suitable display. The external device may comprise an alarm, light, or other indicator, which may be used to signal the practitioner when a certain force has been met or exceeded.

In embodiments, sensors and/or patches may be fabricated using suitable methods, including photolithography, electro deposition, screen printing and the like, and may include materials such as copper, gold, carbon, platinum, or combinations thereof. Sensors may be manufactured using suitable techniques. From the perspective of mechanical feasibility and reliability testing, advanced manufacturing techniques, such as laser cutting, can be used to rapidly manufacture cost-efficient sensor prototypes with complex geometries. For example, conductive polyurethane foam is a versatile material that can be efficiently laser-cut to different thicknesses and geometries; additionally, it can withstand repeated applied mechanical forces while conducting an electrical signal. This allows for ease of redesign and customizability for sensor applications, in addition to speed of production for rapid prototype design and testing. With the use of Computer-Aided-Design (CAD) software to create multiple sensor geometries in one laser-cut batch, laser cutter technologies may be an example of an efficient way to manufacture the sensors in prototype testing for this application. As a subtractive manufacturing technique, laser cutting this simple, yet effective sensor fabrication application may reduce the time, material, and costs that are normally spent manufacturing more complicated sensors.

In embodiments, more than one sensor may be located on a single wire, in various positions along its length. The sensors located along a single wire may be referred to as a “grouping” of sensors. More than one sensor or groupings of sensors may be implanted into a single patient at one or more times. For example, it a sensor or grouping of sensors may be implanted in any of the following locations or combinations thereof: one or a grouping of sensors in each of one or more regions or sections of the spine, such as the cervical, thoracic, thoracolumbar, and/or lumbar regions; one or a grouping of sensors in or near every intervertebral disc, in or near every other disc, in or near every third disc, in or near every fourth disc, or other numbers as well. A single, or multiple patches, may be used in conjunction with a grouping of sensors.

In embodiments, implantable sensors may be implanted using any suitable method, including surgical implantation or inserting them via standard needles and/or cannula. Implantation may be performed in any appropriate clinical setting including a practitioner's office, clinic, or surgical suite. Visual aids such as ultrasound, X-ray radiography, computed tomography (CT), magnetic resonance imaging (MM), or Intraoperative neurophysiological monitoring (TOM), or other methods, or combinations thereof, may be used to properly position sensors, or sensors may be properly positioned using only the practitioner's senses and experience. Sensors may be removed by any suitable method, including surgical removal or pulling them out via wires, which may be connected to them and protrude through the skin to the exterior of the body. Removal may be performed in any appropriate clinical setting including a practitioner's office, clinic, or surgical suite.

In embodiments, implantable sensors may be implanted into a patient before surgery, including several days, weeks, or months before surgery, and may be used to measure loads on the spine during rest or activities (e.g., sitting, standing, walking, stretching). Practitioners may use data collected before surgery for various purposes, including measuring the load characteristics and capabilities of the spine as it exists before surgery. Alternatively, sensors may be implanted just before or at the time of surgery. The sensors may be left in place during surgery and may be used to measure loading that occurs during procedures that are performed. Practitioners may use data collected before, during, and/or after surgery for various purposes, including making real-time use of the data to properly align the spine or joint or anatomical feature. Sensors may be removed during surgery or may remain implanted after surgery, including several days, weeks, or months after surgery, such as during a recovery or rehabilitation period. Practitioners may use data collected before, during, and/or after surgery for various purposes, including determining the load distributions achieved by the surgery and to help to guide rehabilitation of the patient (e.g., the data may be used to help teach a patient better postures for sitting, walking, etc., that may minimize loads or optimize spinal position). In embodiments, implantable sensors may be implanted into a patient who may not currently need alignment surgery, and may be used to measure loads on the spine during rest or activities (e.g., sitting, standing, walking, stretching), and may be used to monitor changes in the condition of the patient.

In embodiments, sensors may be efficiently manufactured into complex structures with easily-manipulatable characteristics for versatility. Sensors may be designed into or combined with tools already used in surgery, such as integrating the sensor with a surgical glove or spinal implant or spinal alignment device. Sensors may be designed with a user-friendly interface.

FIGS. 1A-1C show an exemplary sensor 100 having a “plate-foam-plate” capacitive arrangement. This sensor 100 as well as other configurations, may be assembled and otherwise configured to provide a change in an electrical property and to output that change to a controller or other location for subsequent conversion into a loading experienced by the deployed sensor. The electrical property being monitored may be associated with the capacitive properties of a sensor. In FIG. 1A, a voltage difference is shown to be provided by wires 131 and 132 and the foams, 120 and 150 serve to inhibit movement of the plates 110 and 11 towards each other. As the plates 110 and 111 move towards each other a voltage drop may be detected and may serve to indicate movement of the plates 110, 111 towards each other. This movement may be converted into an applied force experienced by the plates 110, 111 by one or more controllers of embodiments.

FIG. 1A shows a first metal or otherwise conductive plate 110 and a second metal or otherwise conductive plate 111, having a second foam 120 therebetween. First foam 150 is not shown in FIG. 1A, but is shown in FIG. 1B. First foam 150 and second foam 120 have a thickness 140. First foam 150 also has a diameter 141. FIGS. 1A and 1B also show electrical wires 131 and 132 electrically connected to plate 110 and plate 111 respectively. In this illustration, the second foam surrounds the first foam, except at its tops and bottom, where the first foam contacts metal plates 110, 111. In this embodiment, the second foam serves as an insulator to the first foam and a spacer between the metallic plates 110 and 111. Comparatively, the first foam 150 is a conductive polyurethane foam and also serves as a spacer between the metallic plates 110 and 111. Although not illustrated, a sheet or sheets of second foam may also be located between first metal plate 110 and first foam 150, between second metal plate 111 and first foam 150, or both. These additional sheets may, therefore, provide additional spacing between the plates and may also provide additional electrical insulation, if the foams or other spacing materials are insulators. Thus, in embodiments, a first foam or another spacer may be completely encapsulated by a second foam or other spacer.

In FIGS. 1A and 1B, the first foam is shown as a cylinder with a 2.00 cm diameter ratio. However, smaller and larger sensors are contemplated. Exemplary ranges may include 1 mm-10 cm with geometries from round, square, rectangular, ellipse, or disk shaped and combinations thereof. Thus, sensor embodiments may have different shapes and may comprise other configurations as well. In preferred embodiments, a sensor would preferably comprise conductive plates spaced a distance apart, where the plates do not touch and can act as a capacitor when a voltage is placed across the conductive plates.

FIG. 1C shows a load 160 being applied to the sensor 100. In this instance, the load 160 is shown as a compressive force acting opposite to an equal and opposite compressive force 161. Thus, in embodiments, axial or otherwise opposing compressive forces may be placed on a sensor. Under this loading, the sensor may compress under this loading, and under this change in thickness, the sensor may present a measurable change of capacitance, voltage difference, impedance, or other measurable electrical property. This measurable change in electrical property may be subsequently measured and used to determine the normal or non-normal loads being experienced by the sensor. In embodiments the sensors may be calibrated prior to use such that static loads may be known and identified such that a base level may be subtracted from any subsequent active loading on the sensor. This calibration step may be performed when a sensor is first anchored or otherwise deployed at a target location for load detection as well as later on, after deployment, to reset a steading state loading condition or for other reasons as well.

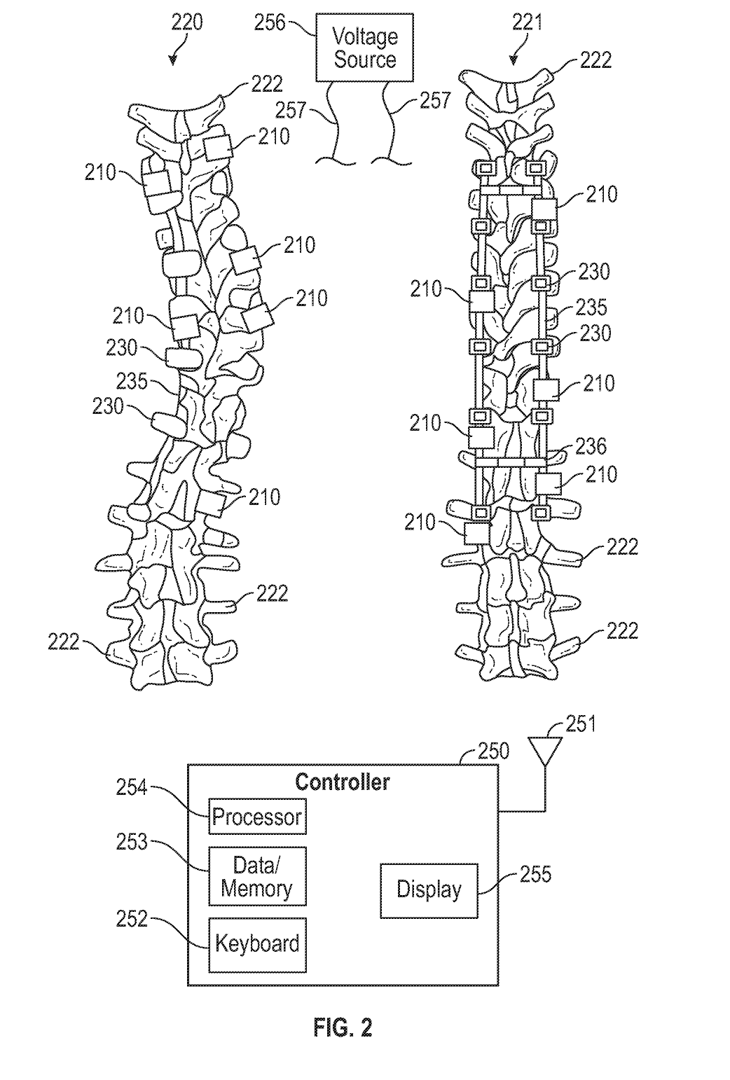

FIG. 2 shows multiple sensors 210, a voltage source 256, and a controller 250, as may be employed in accordance with some embodiments. Also labelled in FIG. 2 are spine 220 and 221, vertebra 222, metallic anchors 230, metallic connector 235, cross-connector236, antenna 251, leads 257, processor 254, data/memory 253, keyboard 252, and display 255. In use, the sensors 210 may be placed at various locations of a spine vertebrae 220 or 221. The sensor 210 may have a stored capacitive charge therein using a chemical reaction or a voltage source to develop the charge. Then, when under load, any change in capacitance, voltage, impedance or other electrical property for the sensors may be measured and used to determine a compression of the sensors as well as a force or pressure being experienced by the sensors.

In some embodiments a communal voltage source 256 may be used to provide a voltage differential to the sensors. In other embodiments, the sensors may each have their own voltage source. As noted above, a self-contained voltage source may be provided by a chemical reaction such as may occur in a battery or other electrical storage device. When embodiments employ a communal voltage source, the voltage source may be connected in parallel to multiple sensors so as to provide the same voltage to each of the sensors connected to the voltage source.

During a procedure, as the spine 220 is straightened, the sensors 210 may send signals to the controller for receipt by its antenna 251. These signals may be used by the controller to determine the forces and/or strains being experienced by each of the sensors 210. In embodiments, the sensors 210 may be individually addressable such that the controller 250 can report to a practitioner the forces being experienced at each sensor location. When a suitable amount of force is identified by the display 255 for a particular vertebra or other subcutaneous location, a practitioner may select a location for an anchor and install it as well as select a connector 235 between anchors 230 and install the connector 235. Thus, a system employing the sensors 210 may provide real-time feedback of forces generated in structural anatomy and may provide useful data for a practitioner to use during surgical reconfiguration or surgical reconstruction or other surgeries or processes. In this and other embodiments, loads being experienced and measured may be stored and used for subsequent analysis and/or display.

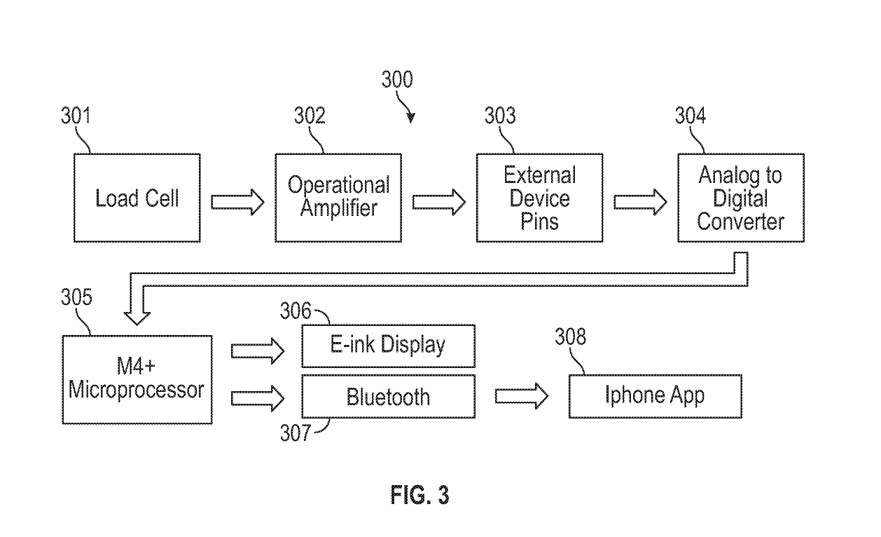

FIG. 3 provides an operational schematic 300 as to how a sensor system may work, in some embodiments. As shown in FIG. 3, a load cell sensor 301 may produce a voltage, such as between 0-10 mV. This voltage may be amplified through an operational amplifier 302, such as an 100V/V amplifier. The now amplified signal, such as 0-1V, may then be externally output through external device pins 303 for receipt by an Analog to Digital Converter (ADC) 304. After the amplified analog signal is converted into a digital value, such as a 16 bit value, it may be post-processed by a microprocessor, such as a M4+ microprocessor 305, to display pound-force or other calculated strain or pressure on an e-ink display 306 or other display. In addition, or alternatively, that same calculated strain or pressure information may be sent through a wireless module, such as Bluetooth module 307, to a smart phone application, such as iPhone application 308, which may display the measurements to an interested user.

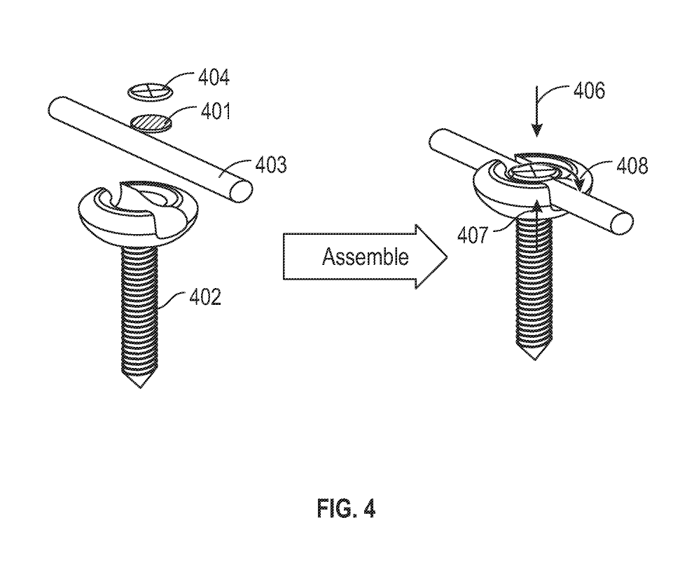

In embodiments, a sensor may be incorporated into a spinal alignment device, such as the anchor shown in FIG. 4. In so doing, a practitioner may be able to determine loads being placed on the alignment devices or other medical implants during implantation as well as afterwards. Exemplary implants may include a rod and screw device, wherein a corrective rod (403 in FIG. 4) may be affixed to a screw anchor 402, and a screw cap 404 may fit over the screw, and a sensor 401 may be positioned between the screw cap and rod. Also labeled in FIG. 4 are applied compressional load to screw cap 406, push-out force from rod to screw cap 407, and applied torsional load to screw cap 408. Embodiments may include a sensor 401 constructed and/or configured to be fitted and/or placed between a screw cap and a corrective rod. The sensor may be shaped and/or sized to fit between a screw cap and a corrective rod. The sensor may be manufactured as a part of a screw cap or rod, or may be placed between the screw cap and rod before or during surgery. The sensor may measure torque, axial force, and/or other forces applied to the screw and/or rod during implantation or afterwards. For example, a sensor of embodiments may assist a surgeon to avoid over-torqueing or under-torqueing a screw or other implant. Also, a sensor can serve to may measure corrective forces between a rod and the spine or other body part of interest and/or may provide quantification of load at a spine segment or other body part of interest. As described in more detail below, when anchors employing sensors are deployed, or when sensors are otherwise affixed placed at a sensing target location, a calibration step may be performed such that background static loads on the sensor may be measured and used to identify active loads on the sensor.

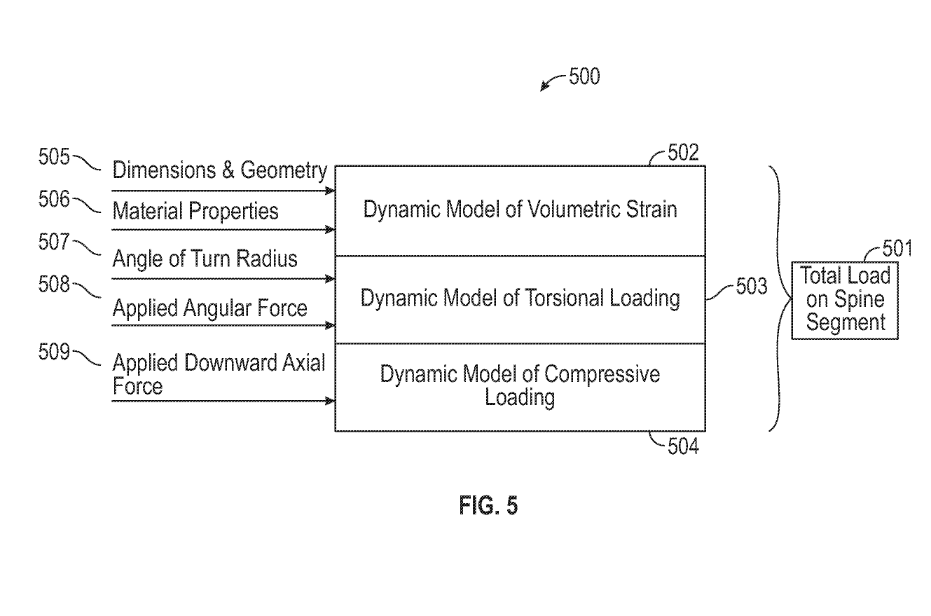

FIG. 5 depicts a functional diagram 500 of exemplary mechanical loading of a load sensor, in accordance with some embodiments. FIG. 5 shows mechanical force components, which add up to the total amount of load which may be experienced by a sensor; the factors (505-509) which may influence each of these components (i.e. volumetric strain 502, torsional loading 503, and compressive loading 504) are listed on the left-hand side; the output of total load which is applied is identified at 501; overall, the mathematical model of mechanical loading is based upon the Beam Column Theory, which is described in Equation A.

The following exemplary mathematical modeling, may be used in embodiments.

Beam Column Theory→Governing Equation Equation A:

Potential Energy=Strain Energy+Potential of Applied Loads

Π=U+V

Where U=Strain Energy, V=Applied Loads
Strain Energy: Compressed Volumetric Strain
Applied Loads: Torsion, Compression

Π=[Compressional Volumetric Strain]+[(Torsion)+(Compression)]Π=[(σ/E)(1−2μ)]+[(F*r*cos θ)+(E*(ΔL/Lo)*A)]

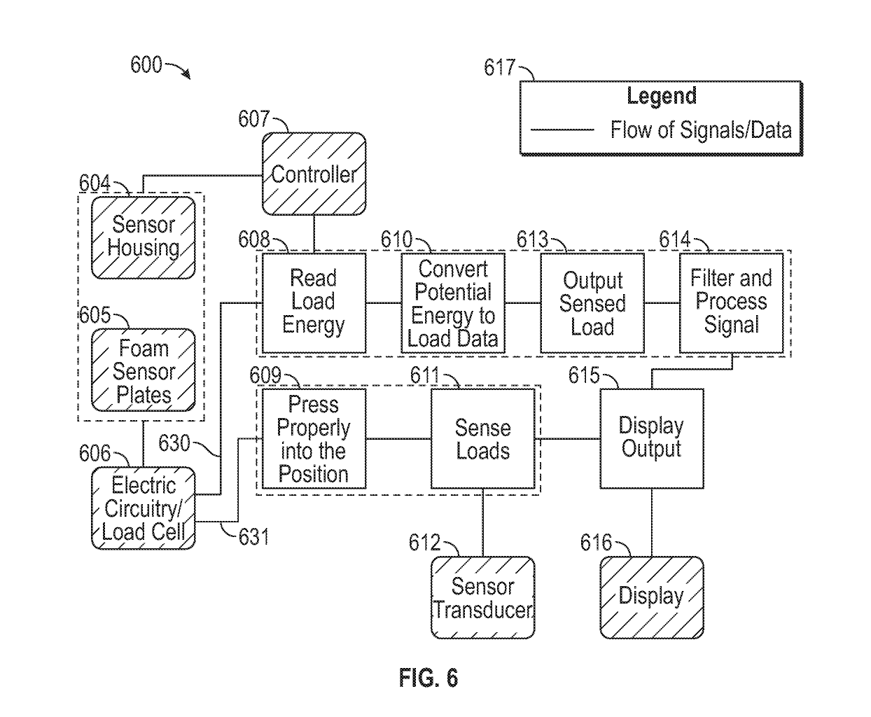

FIG. 6 shows an exemplary schematic 600 of system level component and process flows as may be employed in accordance with some embodiments. Components are signified with shading and resident in rounded corner boxes of FIG. 6 while process steps are shown in unshaded boxes of FIG. 6. Grouped components or processes are shown within dashed surrounding lines. Legend 617 identifies that the flow of signals/data between components, as well as amid process steps, as may be employed in embodiments, is identified with a solid bridging line. Process steps that may be employed when sensing compression of a deployed sensor are shown at 608, 610, 613, and 614 while process steps that may be employed during calibration cycles of a sensor are shown at 609 and 611. The process steps during sensing compression (608, 610, 613, and 614) as well as the calibration steps (611) may be performed by the patch or controller 607, by the electric circuitry 606, as well as by both in certain embodiments. Also labelled in FIG. 6 are display output 615, sensor transducer 612, and display 616.

In embodiments, design considerations for an exemplary sensor system may include providing sufficient structure around a sensor to be employed, providing power to a sensor to be employed, and performing command and control of the sensor or other system functions. The power being provided to the sensor in embodiments, may be internally provided as with a battery or chemical reaction as well as be provided from an external source through wires, induction, or other power transfer technique. In FIG. 6, a load cell is provided with electric circuity 606 and is resident outside of the sensor housing 604 and foam, sensor, and plates 605. However, in embodiments, as mentioned above, the electric circuitry and/or load cell may be resident with the sensor housing 604 and foam, sensor, and plates 605. Whether resident with the sensor housing 604 or not, the electric circuitry 606 may be employed during calibration processes, as shown at 609 and related processes 611, as well as with deployed load sensing, as is shown at 608 and related processes 610, 613, and 614.

The sensor housing 604, foam & sensor plates 605 and electrical circuitry/load cell 606 may be mechanically surrounded or otherwise mechanically secured in embodiments. Battery power may be provided to electric circuitry and a load cell 606 of embodiments. Command and control signaling may also be sent to and from electrical circuitry/load cells 606 of embodiments. Controller 607 is shown in FIG. 6 as being positioned to receive load energy signals from the electric circuitry 608. Process box 610 shows that voltage potential may be converted to load data and then this data may be output at 613 as a sensed load. Subsequent filtering and signal processing may be carried out in embodiments, as shown at process box 614 and subsequently displayed as shown at process box 615 and display 616. Component box 612 shows that a transducer may also be employed in embodiments and the loads may be sensed during a calibration cycle by process step 611. The path for data flow to and from the electric circuitry 606 is shown at 630 and 631.

Table A sets forth specifications and target values for an electrochemical component of a sensor, as may be employed in embodiments. Other target values may also be employed.

TABLE E

| Specification | Target Value |
|---|---|
| Sensitivity | >1.14 kPa⁻¹ |
| Response Rate | <17 ms |
| Simplicity in Fabrication | Yes (Binary) |
| Size | Microsize |
| Reproducibility | Yes (Binary) |
| Lifetime | 500 Loading - Unloading Cycles |
| Biocompatibility | Minimally Irritating |

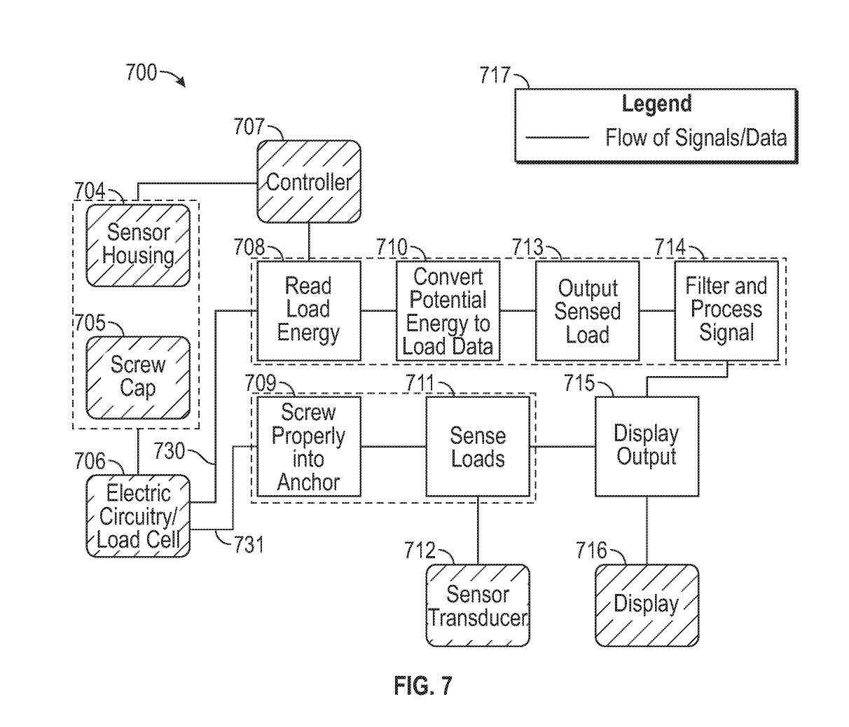

Like FIG. 6, FIG. 7 shows an exemplary schematic 700 of operational sharing within the general flow of a sensor's design and operation, as well as applicable components, as may be employed in accordance with some embodiments. A notable difference between FIG. 6 and FIG. 7 is that in FIG. 6 the sensor is being calibrated after being pressed into position, while the sensor in FIG. 7 is being calibrated after being screwed into position. Thus, FIG. 6 shows that sensors may be deployed and held in place with compression, as may be done with hand held tools, gloves, non-anchored sensors, while FIG. 7 shows that sensors may be deployed and held in place through torsional anchoring, e.g., screws as in FIG. 4.

Components are shaded and resident in rounded corner boxes of FIG. 7 while process steps are shown in unshaded boxes of FIG. 7. Grouped components or processes are shown within dashed surrounding lines. Legend 717 identifies the flow of signals/data for this schematic and uses the same solid line key as shown in FIG. 6. Design of an exemplary sensor may include providing sufficient structure around the sensor, providing power to the sensor, and performing command and control of sensor functions. A sensor housing 704 may be used to provide surrounding structure for the sensor and may include a screw cap 705 when a torsional anchored sensor is employed in embodiments. Electrical circuitry 706 may be battery powered and may be configured for control and sensing as described herein. A sensor housing 704 may be sufficient to provide surrounding structure for the sensor and may include a screw cap 705 while electrical circuitry and a load cell 706 may be configured for control and sensing as described herein. Process steps that may be employed when sensing compression of a deployed sensor are shown at 708, 710, 713, and 714 while process steps that may be employed during calibration cycles of a sensor are shown at 709 and 711. The process steps during sensing compression (708, 710, 713, and 714) as well as the calibration steps (711) may be performed by the patch or controller 707, by the electric circuitry 706 as well as by both in certain embodiments. Also labelled in FIG. 7 are display output 715, sensor transducer 712, and display 716. The path for data flow to and from the electric circuitry 706 is shown at 730 and 731.

In use, calibration steps 709 and 711 may be performed once a load cell is positioned at a target location for pressure sensing. The calibration cycle, here are well as in other embodiments, may be performed to designate a zero state for the sensor. In other words, the sensor may be under static loads created by its anchoring and these loads may be identified during calibration in order to determine a base loading that can be subtracted from any active loading experienced by the anchored sensor. An active loading cycle may be sensed as is shown by box 708, and received by controller 707 as well as by electric circuitry 706. Using this read energy load the controller 707 and or the electric circuitry 706 may be configured to determine a load from received data. This load may be determined by converting a voltage change or capacitance change or inductance change sensed into a force value. A conversion from an electrical property change to a force reading may be specific for a certain sensor and may be linear as well as nonlinear. In other words, a linear relationship between changes in electrical properties and applied loads may exist for a first sensor and a nonlinear relationship between changes in electrical properties and applied loads may be nonlinear for a second sensor. Sensors of embodiments may also demonstrate hybrid properties whereby loading and electric outputs are linear for a first portion of a loading curve and nonlinear for a second portion of a loading curve. The controller 707 as well as the electric circuitry 706 may each be configured to output the sensed load as is shown in process box 713 and filter and process signals from the various components of the system as is shown at box 714. Display output box shows that processes may be included in embodiments whereby a module in a controller 707 or electric circuity 706 or other location may be configured to accept filtered and processed signals or otherwise sensed loads and output the data to a readable display 716. The sensor transducer 712 of FIG. 7 may be employed for purposes of calibrating a deployed sensor and, for example, identifying static loads created by anchoring of the deployed sensor.

Table B presents certain exemplary metrics that may be considered in embodiments of a sensor, as well as exemplary target values for those metrics, which may be achieved in some embodiments.

TABLE B

| Specification | Performance Value |
|---|---|
| Compression Range | 0-350N |
| Mechanical Reliability | >50 Cyclic Compressions |
| Signal Sensing Accuracy | 90% |
| Pressure Sensing Accuracy | Microamperes (μA) |
| Alert/Feedback System | Binary Indicator (e.g., LED or Sound) |
| Manufacturing Cost | $97-$250 |

In embodiments, a sensor may be incorporated into a surgical glove or other medical device. The sensor may be shaped and/or sized to fit on a glove. The sensor may comprise an electrode partially or entirely coated in foam and/or may comprise a plate-foam-plate arrangement. The sensor may be manufactured as a part of a glove, or may be placed on or affixed to the glove before or during surgery. The sensor may be placed on or affixed to the glove using suitable means, such as adhesives, Velcro, a strap, or other mechanical means. The sensor may measure torque (angular force), downward (axial) force, and/or other forces applied by the practitioner wearing the glove. The sensor may assist a surgeon to avoid over-torqueing or under-torqueing spinal alignment devices. The sensor may comprise an electrode partially or entirely coated in foam. The force range requirement of the sensor for may be approximately 240-500N. The foam may comprise or be impregnated with electroactive moieties. The sensor may comprise a power source, such as a battery, a device that converts bodily motion to electrical power, or a device that converts light to electrical power. The sensor may comprise a device that receives power wirelessly. The sensor may be connected via a wired or wireless connection to an external controller, such as a cell phone, computer, or dedicated device, which may provide power to the sensor, may collect voltage changes, or other data from the sensor, may convert data from the sensor to Newtons other measures of force, and may display the raw or converted data. Sensor systems may also comprise a display, which may display raw or processed data from one or more sensors. The sensor systems also comprise an alarm, light, or other indicator, which may be used to signal the practitioner when a certain force has been met or exceeded. In embodiments, a sensor system may be used with many different types of spinal surgeries. In embodiments, a sensor system may be used with many different types of corrective spinal surgeries. In embodiments, a sensor system may be used with many different types of scoliosis surgeries. In embodiments, a sensor system may be used with scoliosis surgeries that employ hooks and wires as well as posterior rod and screw techniques and/or spacing and alignment technologies. In embodiments, the sensor system may be used with other types of implant surgeries (e.g., hip/knee replacements) that require use of force and/or measurement of force. In embodiments, sensor systems may also be used to evaluate and/or optimize prosthetic sensor-motor capabilities.

While embodiments have been illustrated herein, it is not intended to restrict or limit the scope of the appended claims to such detail. In view of the teachings in this application, additional advantages and modifications will be readily apparent to and appreciated by those having ordinary skill in the art. Accordingly, changes may be made to the above embodiments without departing from the scope of the disclosure.

Various features, steps, processes, components, and subcomponents may be employed in certain embodiments. These features, steps, processes, components, subcomponents, partial steps, systems, devices, etc. may be adjusted, combined and modified in various fashions and various ways among and between the teachings and figures provided herein, as well as in other ways not specifically described herein but consistent with the teachings and discussion of this disclosure unless otherwise indicated herein or otherwise clearly contradicted by context. All methods described herein can be performed in any suitable order unless otherwise indicated herein or otherwise clearly contradicted by context.

The terminology used herein is for the purpose of describing particular embodiments only and is not intended to be limiting. As used herein, the singular forms “a,” “an” and “the” are intended to include plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The use of any and all examples, or exemplary language (e.g., “such as”) provided herein, is intended merely to better illuminate and does not pose a limitation on scope unless otherwise claimed. No language in the specification should be construed as indicating any non-claimed element as essential.

As used herein, the terms “about” or “approximately” in reference to a recited numeric value, including for example, whole numbers, fractions, and/or percentages, generally indicates that the recited numeric value encompasses a range of numerical values (e.g., +/−5% to 10% of the recited value) that one of ordinary skill in the art would consider equivalent to the recited value (e.g., performing substantially the same function, acting in substantially the same way, and/or having substantially the same result). As used herein, the terms “about” or “approximately” in reference to a recited non-numeric parameter generally indicates that the recited non-numeric parameter encompasses a range of parameters that one of ordinary skill in the art would consider equivalent to the recited parameter (e.g., performing substantially the same function, acting in substantially the same way, and/or having substantially the same result).

Recitation of ranges of values herein are merely intended to serve as a shorthand method of referring individually to each separate value falling within the range, unless otherwise indicated herein, and each separate value is incorporated into the specification as if it were individually recited herein.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

In embodiments, a “patient” may be an animal in need of spinal correction, joint replacement or correction, or other surgeries that require use of force and/or measurement of force. The patient may be in need of spinal correction for spinal misalignment. The spinal misalignment may be congenital, caused by aging, or caused by injury. The patient may be in need of correction of scoliosis. The patient may be an animal where the animal may be a mammal, a reptile, or a bird. Still further, the animal may be a companion animal, an agricultural animal, a laboratory animal, a zoological animal, or a wild animal. The animal may be a human. The patient may be embryonic or fetal, infant, juvenile or pediatric, adolescent, young adult, adult, or geriatric. The age of the patient may range from pre-birth to 100 or more years old. The patient subject may be male, female, androgynous, or inter-sexual and of any ethnic origin.

Certain embodiments may be implemented as a computer process, a computing system or as an article of manufacture such as a computer program product of computer readable media. The computer program product may be a computer storage medium readable by a computer system and encoding computer program instructions for executing a computer process.

The corresponding structures, material, acts, and equivalents of any means or steps plus function elements in the claims are intended to include any structure, material or act for performing the function in combination with other claimed elements. The description of certain embodiments has been presented for purposes of illustration and description, but is not intended to be exhaustive or limited to the form disclosed. Many modifications and variations will be apparent to those of ordinary skill without departing from the scope and spirit of the disclosure. These embodiments were chosen and described in order to best explain the principles of the disclosure and practical application, and to enable others of ordinary skill in the art to understand possible embodiments with various modifications as are suited to the particular use contemplated.

What is claimed is:

1. An anatomical force sensor system, the sensor system comprising:
a first electrically conductive plate;
a second electrically conductive plate, the first conductive plate and the second conductive plate spaced a distance apart from each other and not touching each other;
a first compressible foam disposed between the first electrically conductive plate and the second electrically conductive plate, the first compressible foam having a first thickness in a first compression state and a second thickness in a second compression state;
a second compressive foam disposed about the first compressive foam, the second compressive foam providing electrical insulation to the first compressive foam;
a voltage source electrically coupled to the first electrically conductive plate and the second electrically conductive plate; the voltage source configured to provide a voltage potential between the first conductive plate and the second conductive plate; and
a controller, the controller configured to sense changes in capacitance between the first electrically conductive plate and the second electrically conductive plate when the conductive plates are placed within a body of a patient and use these sensed changes in capacitance to determine a change in distance between the first conductive plate and the second conductive plate.

2. The anatomical force sensor system of claim 1, wherein the first compressible foam and the second compressible foam each have a top and a bottom and at least one side, the at least one side of the first compressible foam surrounded by the second compressible foam.

3. The anatomical force sensor system of claim 2, wherein the top of the first compressible foam and the top of the second compressible foam are adjacent to the first electrically conductive plate and the bottom of the first compressible foam and the bottom of the second compressible foam are adjacent to the second electrically conductive plate.

4. The anatomical force sensor system of claim 3, wherein the top of the first compressible foam and the top of the second compressible foam touch the first electrically conductive plate and the bottom of the first compressible foam and the bottom of the second compressible foam touch the second electrically conductive plate.

5. The anatomical force sensor system of claim 1, wherein the first compressible foam is cylindrical and the second compressible foam is polygonal.

6. The anatomical force sensor system of claim 1, wherein the voltage source is electrically coupled to the first electrically conductive plate with a wire and electrically coupled to the second conductive plate with a wire.

7. The anatomical force sensor system of claim 1, wherein the first compressible foam comprises electroactive moieties, wherein the first electrically conductive plate and the second electrically conductive plate are parallel, and wherein the voltage source is sized to limit a capacitive charge of no greater than 100 millivolts from being developed between the first electrically conductive plate and the second electrically conductive plate.

8. The anatomical force sensor system of claim 1, wherein the controller is further configured to determine, using the change in distance between the first electrically conductive plate and the second electrically conductive plate, real-time compressive forces experienced by the first electrically conductive plate or the second electrically conductive plate or both.

9. The anatomical force sensor system of claim 1, wherein the first electrically conductive plate, the second electrically conductive plate, the first compressible foam, and the second compressive foam are resident within an implantable biocompatible sensor.

10. The anatomical force sensor system of claim 1, wherein the first electrically conductive plate, the second electrically conductive plate, the first compressible foam, the second compressive foam, and the voltage source, are resident within an implantable biocompatible sensor.

11. An implantable sensor system, the sensor system comprising:
an implantable sensor; and
a controller,
wherein the implantable sensor comprises:
a first electrically conductive plate;
a second electrically conductive plate, the first electrically conductive plate and the second electrically conductive plate spaced a distance apart from each other and not touching each other;
a first compressible foam disposed between the first electrically conductive plate and the second electrically conductive plate, the first compressible foam comprising electroactive moieties, the first compressible foam having a first thickness in a first compression state and a second thickness in a second compression state; and
a second compressive foam disposed about the first compressive foam, the second compressive foam providing electrical insulation to the first compressive foam,
wherein the controller is configured to sense changes in capacitance between the first electrically conductive plate and the second electrically conductive plate and use these sensed changes in capacitance to determine a real-time change in distance between the first electrically conductive plate and the second electrically conductive plate when the conductive plates are resident within a body of a patient.

12. The implantable sensor system of claim 11, further comprising:
a voltage source electrically coupled to the first electrically conductive plate and the second electrically conductive plate; the voltage source configured to provide a voltage potential between the first electrically conductive plate and the second electrically conductive plate.

13. The implantable sensor system of claim 11, wherein the first compressible foam and the second compressible foam each have a top and a bottom and at least one side, the at least one side of the first compressible foam surrounded by the second compressible foam.

14. The implantable sensor system of claim 11, wherein the top of the first compressible foam and the top of the second compressible foam are adjacent to the first electrically conductive plate and the bottom of the first compressible foam and the bottom of the second compressible foam are adjacent to the second electrically conductive plate.

15. The implantable sensor system of claim 11, wherein the first compressible foam comprises electroactive moieties, wherein the first electrically conductive plate and the second electrically conductive plate are parallel, and wherein the voltage source is sized to limit a capacitive charge of no greater than 1,000 millivolts from being developed between the first electrically conductive plate and the second electrically conductive plate.

16. The implantable sensor system of claim 11, wherein the controller is further configured to determine, using the change in distance, real-time compressive forces experienced by the first electrically conductive plate or the second electrically conductive plate or both.

17. A method for measuring forces exerted on one or more vertebrae, the method comprising:
receiving a data signal at a controller; and
providing a determination of real-time force exerted on an implanted anatomical sensor,
wherein the data signal is generated by a sensor positioned adjacent one or more vertebrae of a patient,
wherein the implanted anatomical sensor comprises:
a first electrically conductive plate;
a second electrically conductive plate, the first conductive plate and the second conductive plate spaced a distance apart from each other and not touching each other;
a first compressible foam disposed between the first electrically conductive plate and the second electrically conductive plate, the first compressible foam comprising electroactive moieties, the first compressible foam having a first thickness in a first compression state and a second thickness in a second compression state; and
a second compressive foam disposed about the first compressive foam, the second compressive foam providing electrical insulation to the first compressive foam,
wherein the controller is configured to sense changes in capacitance between the first electrically conductive plate and the second electrically conductive plate and use these sensed changes in capacitance to determine a change in distance between the first conductive plate and the second conductive plate.

18. The method for measuring forces exerted on one or more vertebrae of claim 17 wherein the first compressible foam and the second compressible foam each have a top and a bottom and at least one side, the at least one side of the first compressible foam surrounded by the second compressible foam.

19. The method for measuring forces exerted on one or more vertebrae of claim 17, wherein the top of the first compressible foam and the top of the second compressible foam are adjacent to the first electrically conductive plate and the bottom of the first compressible foam and the bottom of the second compressible foam are adjacent to the second electrically conductive plate.

20. The method for measuring forces exerted on one or more vertebrae of claim 17, wherein the controller is further configured to determine, using the change in distance, real-time compressive forces experienced by the first electrically conductive plate or the second electrically conductive plate or both.
